(19) United States
(12) Patent Application Publication
(10) Pub. No.: US 2002/0137098 A1
(43) Pub. Date: Sep. 26, 2002

(54) Methods for detecting retinopathy of prematurity

(75) Inventors: Kathryn M. Brady-McCreery, Houston, TX (US); Charles J. McCreery, Houston, TX (US)

Correspondence Address: MUETING, RAASCH & GEBHARDT, P.A., P.O. BOX 581415, MINNEAPOLIS, MN, 55458, US

(73) Assignee: Board of Regents, The University of Texas System, Austin, TX

(21) Appl. No.: 10/029,116

(22) Filed: Oct. 19, 2001

Related U.S. Application Data
(60) Provisional application No. 60/242,291, filed on Oct. 20, 2000.

(51) Int. Cl. G01N 33/53; G01N 33/567

(52) U.S. Cl. 435/7.1; 435/7.21

(57) ABSTRACT

The present invention provides methods for determining is a premature infant is at-risk of developing severe Retinopathy of Prematurity by detecting the level of vascular endothelial growth factor in a biological sample obtained from the premature infant. Also provided by the present invention is a kit for determining whether a premature infant is at risk of developing severe Retinopathy of Prematurity.

### CONTINUING APPLICATION DATA

This application claims the benefit of U.S. Provisional Application Serial No. 60/242,291, filed Oct. 20, 2000, which is incorporated by reference herein.

### BACKGROUND

Retinopathy of Prematurity (ROP) is a leading cause of blindness in premature infants and its incidence in the United States is rising with increased survival of extremely premature infants (Arch. Ophthalmol., 106, 471-479 (1988); Paysse. J. of Am. Assoc. Pediat. Ophthalmol. Strabismus, 3, 234-240 (1999)). This disease results in the growth of new blood vessels (neovascularization) in the eye of premature infants which in turn pull on retinal tissues and cause retinal detachment. Retinal detachment surgery in these premature eyes is complicated, and vision is often lost despite the most heroic efforts.

ROP typically develops between about 34 weeks and 40 weeks after conception. The peak incidence of severe ROP is at about 37 weeks. Screening programs are present in most hospital neonatal intensive care units. Screening an infant for the presence of ROP requires the direct observation of the infant's retinas using an examination light under topical anesthesia.

ROP is a complex and often progressive disease process. The level of progression is expressed as various stages (stages 0-3, and threshold), and the hallmarks of each stage is described by the Committee for the Classification of Retinopathy of Prematurity (Arch. Ophthalmol., 102, 130-1134 (1994)). Stage 1 ROP and Stage 2 ROP represent mild disease and stage 3 ROP represents neovascular proliferation which may result in retinal traction and blindness. Threshold ROP is an amount of stage 3 ROP that is thought to require immediate treatment.

Vascular endothelial growth factor (VEGF) is a family of potent cytokines which act to induce new blood vessel formation (angiogenesis) and markedly increase microvascular permeability. During fetal development, VEGF is expressed in multiple embryonic and fetal tissues, with the highest levels found in the lung, kidney and heart. It is also found in placental tissues and its concentration increases with advancing gestation (Plate et al., J. Neuro-Oncol., 35, 365-72 (1997)). Robbins et al. (Growth Factors, 16, 1-9 (1998)) demonstrated VEGF receptor protein being concentrated in preretinal neovascular growths in an animal model of ROP. Other investigators demonstrated a continuous intense band of VEGF mRNA expression across the peripheral avascular retina adjacent to areas of retinal neovascularization (Dorey et al., Arch Ophthalmol., 114, 1210-1217 (1996)). VEGF has been identified in membranes surgically removed in patients with proliferative diabetic retinopathy, proliferative vitreo retinopathy and macular pucker (Armstrong et al., Ophthalmologica, 212, 410-414 (1998)) and has been found in increased concentrations in the eyes of patients with proliferative diabetic retinopathy (Boulton et al., British J. Ophthalmol., 82, 561-568 (1998)). The use of recombinant formulations of angiogenic growth factors such as VEGF to augment collateral artery development by stimulation of capillary growth in animal models of myocardial and hindlimb ischaemic has also been established. Research studies are currently underway evaluating the use of VEGF to promote collateral coronary artery formation in ischaemic heart disease in humans (Baumgartner and Isner, Vasa, 27, 201-206 (1998)).

### SUMMARY OF THE INVENTION

The present invention represents an advance in the art of determining whether an infant is at-risk for developing severe retinopathy of prematurity (ROP). As used herein, “severe ROP” refers to ROP that is potentially blinding, and includes stage 3 ROP and threshold ROP. The present invention is based on the observation that when the level of VEGF in the blood of premature infants was plotted as a function of the number of weeks post-conceptual age, the levels of VEGF in the infants that did not later develop severe ROP generally decreased from about 32 weeks post-conceptual age to about 40 weeks post-conceptual age. In contrast, in infants that later developed severe ROP, the levels of VEGF were highest at about 36 weeks to about 37 weeks post-conceptual age.

The present invention is directed to a method for determining if a premature infant is at-risk of developing severe ROP. The method includes detecting the level of VEGF in a first biological sample and a second biological sample obtained from a premature infant, where the second biological sample is obtained after the first biological sample. Preferably, the biological sample is blood, serum, or plasma. The levels of VEGF in the biological samples are compared. Preferably, the first biological sample is obtained when the premature infants' post-conceptual age is at about 32 weeks. Preferably, the second biological sample is obtained about a week after the first biological sample is obtained. A premature infant who is not at-risk of developing severe ROP has a lower level of VEGF in the second biological sample, and a premature infant who is at-risk of developing severe ROP has a higher level of VEGF in the second biological sample.

Another aspect of the present invention provides a method for determining if a premature infant is at-risk of developing severe ROP, where the method includes detecting the level of VEGF in a first biological sample obtained from a premature infant, where the first biological sample is obtained from the premature infant by a post-conceptual age of about 35 weeks. Additional biological samples are obtained from the premature infant and the level of VEGF in each of the additional biological samples is detected. The additional biological samples are obtained at intervals of about 1 week following the first biological sample. The levels of VEGF in each of the biological samples is compared. A premature infant who has a level of VEGF that is highest at about 36 weeks to about 37 weeks post-conception age is at-risk of developing severe ROP, and a premature infant who has a level of VEGF that is not highest at about 36 weeks to about 37 weeks post-conception age is at-risk of developing severe ROP. Preferably, the biological sample is blood, serum, or plasma.

Also provided by the present invention is a kit for determining whether a premature infant is at-risk of developing severe ROP. The kit includes reagents for measuring the level of VEGF in a biological sample and packaging material that includes instructions indicating how to determine whether a premature infant is at-risk for developing severe ROP.

### BRIEF DESCRIPTION OF THE FIGURES

FIG. 1. Time serum sample obtained (28 weeks, 32 weeks, 36 weeks, or 40 weeks post-conception age) from premature infants verses levels of vascular endothelial growth factor (VEGF) in picograms per milliliter (pg/ml). Stage 0, 1, 2, 3, and Threshold refers to the ocular status of the patients.

### DETAILED DESCRIPTION OF PREFERRED EMBODIMENTS OF THE INVENTION

The present invention provides methods for determining whether an infant is at-risk for developing severe retinopathy of prematurity (ROP). A “premature infant” is an infant having a gestation time of less than about 28 weeks, or a birth weight of less than about 1500 grams, preferably, less than about 1,250 grams, more preferably, less than about 1,000 grams. The method includes detecting the level of vascular endothelial growth factor (VEGF) present in at least two biological samples obtained from a premature infant. As used herein, a “biological sample” refers to a sample of tissue or fluid isolated from a premature infant, including but not limited to, for example, blood, plasma, serum, and urine, preferably, blood, plasma, or serum. The biological sample may be used immediately, or stored at about −20° C. until used.

Typically, a first biological sample is obtained as soon after birth as the health of premature infant permits. A second biological sample is obtained about a week after the first biological sample, preferably, about 2 weeks. The amount of biological sample required can vary depending on the assay used to detect the level of VEGF, and the amount obtained should not exceed guidelines for the collection of biological samples from premature infants. Such guidelines are known in the art. Optionally and preferably, additional biological samples are obtained at weekly intervals, preferably, every other week. The concentration of VEGF in each biological sample is determined and compared. In one aspect of the invention, when a biological sample is obtained by about 32 weeks, preferably, at about 32 weeks, post-conceptional age or later, and the concentration of VEGF in the next biological sample is lower, the infant is considered to be not at-risk based on the VEGF concentration. When a biological sample is obtained by about 32 weeks, preferably, at about 32 weeks, post-conceptional age or later, and the concentration of VEGF in the next biological sample is higher, the infant is considered to be at-risk based on the VEGF concentration.

In another aspect of the invention, the first biological sample is obtained from the premature infant by a post-conceptual age of about 35 weeks and additional biological samples are later obtained at about weekly intervals. The level of VEGF in each biological sample is determined and compared. A premature infant having a level of VEGF that is greatest at about 36 weeks to about 37 weeks post-conceptual age is considered to be at-risk based on the VEGF concentration, and a premature infant having a level of VEGF that is not greatest at about 36 weeks to about 37 weeks post-conceptual age is considered to be not at-risk based on the VEGF concentration.

VEGF, also known as vascular permeability factor (VPF) and vasculotropin (see, for instance, Ferrara and Henzel, Biochem. Biophys. Res. Commun., 161, 851 -858 (1989), Senger et al., Science., 219, 983 (1983), and Plouet et al., EMBO J., 8, 3807 (1989)), is an endothelial cell-specific growth factor. Several forms of VEGF, including VEGF-A, VEGF-B, VEGF-C, and VEGF-D have been identified (see, for instance, Achen et al., U.S. Pat. No. 6,235,713). The method used to measure the concentration of VEGF is not intended to be a limiting aspect of the present invention, and the assay may detect any form of VEGF that is present in the biological sample. Preferably, the assay detects one of the 5 forms of VEGF-A, more preferably, VEGF₁₆₅.

An assay for detecting the presence of VEGF may be based on the activity of VEGF. For instance, the presence of VEGF in a biological sample may be evaluated by measuring the proliferation of endothelial cells after exposure to the biological samples. Examples of such assays are known to the art (see, for example, Claffey et al., Biochem. Biophys. Acta., 1246, 1-9 (1995), Ferrara and Henzel, Biochem. Biophys. Res. Commun., 161, 851-858 (1989), and Gospodarowicz et al., Proc. Natl. Acad. Sci. USA, 86, 7311-7315 (1986)). Preferably, the assay for VEGF is based on the use of antibody specific for VEGF. As used herein, an antibody that is “specific for VEGF” is an antibody that interacts only with the epitope of the antigen that induced the synthesis of the antibody, or interacts with a structurally related epitope. “Epitope” refers to the site on an antigen to which specific B cells and/or T cells respond so that antibody is produced. The antibody may be polyclonal or monoclonal, preferably, monoclonal. When polyclonal antibody is used, preferably it is produced using human VEGF as antigen; however, due to the highly conserved nature of VEGF between many animals, polyclonal antibody to VEGF isolated from other animals, including, for instance, bovine or rat VEGF, may be used. Methods for obtaining VEGF that can be used to produce antibodies are known to the art. Laboratory methods for producing, characterizing, and optionally isolating polyclonal and monoclonal antibodies are known in the art (see, for instance, Harlow E. et al. Antibodies: A laboratory manual Cold Spring Harbor Laboratory Press, Cold Spring Harbor (1988). Various formats for using antibody to detect antigen may be used, including radioimmunoassay and enzyme immunoassay (also referred to in the art as enzyme linked immunosorbant assay). enzyme immunoassays are available under the trade names COLORIMETRIC (EIA) (Assay Designs, Inc., Ann Arbor, Mich.) and QUANTIKINE (R & D Systems, Minneapolis, Minn.).

In addition to the methods described herein, other risk factors may be used by a person of skill in the art in determining whether a premature infant is at-risk for developing severe ROP. Other risk factors include, for instance, gestational age, birth weight, exposure to supplemental oxygen, and race. Other potential risk factors include elevated blood carbon dioxide levels, anemia, intraventricular hemorrhage, respiratory distress syndrome, chronic hypoxia in utero, multiple spells of apnea or bradycardia, the use of mechanical ventilation, and seizures. Premature infants who are considered to be at-risk of developing severe ROP are closely monitored for the onset of severe ROP. Such monitoring typically includes screening by direct observation of the infant's retinas.

The present invention also provides a kit for diagnosing whether a premature infant is at-risk for developing severe ROP. The kit includes an antibody to VEGF in a suitable packaging material in an amount sufficient for at least one assay. Optionally, other reagents such as buffers and solutions needed to practice the invention are also included. Instructions for use of the antibody are also included.

As used herein, the phrase “packaging material” refers to one or more physical structures used to house the contents of the kit. The packaging material is constructed by known methods, preferably to provide a sterile, contaminant-free environment. The packaging material has a label which indicates that the antibody can be used for detecting the presence and concentration of VEGF and how to diagnose whether a premature infant is at-risk. As used herein, the term “package” refers to a solid matrix or material such as glass, plastic, paper, foil, and the like, capable of holding within fixed limits an antibody. Thus, for example, a package can be a microtiter plate well to which an antibody that specifically binds VEGF has been affixed. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and how to interpret the results.

The present invention is illustrated by the following examples. It is to be understood that the particular examples, materials, amounts, and procedures are to be interpreted broadly in accordance with the scope and spirit of the invention as set forth herein.

### EXAMPLES

Patients and Methods

Twenty-eight patients born at or less than 28 weeks gestation and less than or equal to 1,250 grams were included in the study. Serum samples were obtained at 28, 32, 36, and 40 weeks gestation wherever possible. Blood (0.5 cc) was collected in an aprotonin tube and placed on ice immediately. The blood was centrifuged within 15 minutes and stored at −20° C. until used. VEGF levels were determined as suggested by the manufacturer using the VEGF QUANTIKINE Human VEGF Immunoassay, Catalog Number DVEOO, from R & D Systems (Minneapolis, Minn.).

Other data collected included the ROP stage (stage 1 ROP, stage 2 ROP, stage 3 ROP, and threshold ROP), intraventricular hemorrhage (IVH), bronchopulmonary dysplasia (BPD), sepsis and necrolizing enterocolitis (NEC). Stage 1 ROP and Stage 2 ROP represent mild disease and stage 3 ROP represents neovascular proliferation which may result in retinal traction and blindness. Threshold ROP is is an amount of stage 3 ROP that is thought to require immediate treatment. The presence of IVH was determined by head ultrasonography. BPD and NEC were determined by physical examination performed by a neonatologist.

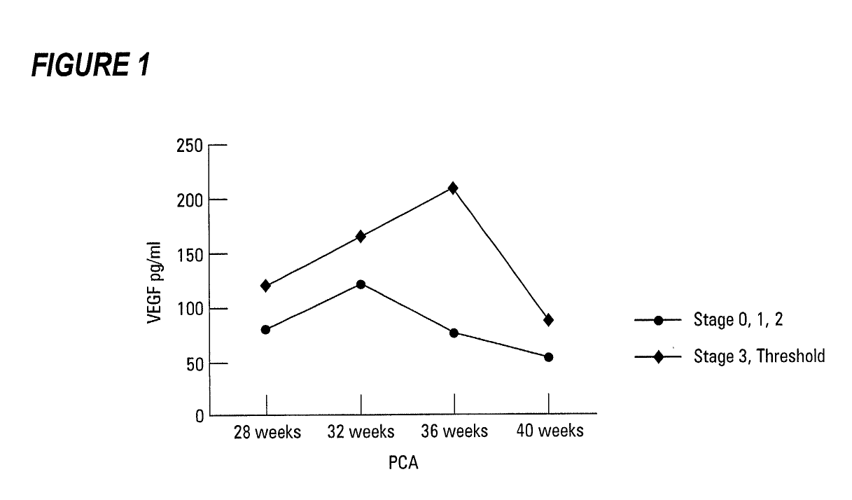

Results The VEGF data were analyzed using a random effects model for longitudinal data. This model allows for efficient analysis of serial observations. VEGF was measured at each of four time points. A total of 59 samples were analyzed. The post conceptual age (PCA) at each time point was recorded to the nearest day. The patients were classified by ROP stage and divided into two groups; those with stage 0, 1 and 2 and a second group with stage 3 or threshold disease. The results are presented in Table 1, and graphed in FIG. 1.

TABLE 1

| | Average VEGF level pg/ml ± SD at weeks post-conceptual age | | | |
|---|---|---|---|---|
| Ocular Status | VEGF 28 wks | VEGF 32 wks | VEGF 36 wks | VEGF 40 wks |
| Stage 0, 1 & 2 | 79.5 ± 44 | 119 ± 76 | 73 ± 44 | 47.7 ± 18 |
| Stage 3, threshold | 117 ± 144 | 159 ± 82 | 204 ± 174 | 84 ± 69 |

pg/ml, picogram/milliliter.

The model was fit with a linear and quadratic term for PCA and a term representing threshold status. Tests that the curves were parallel between the threshold and non-threshold patients indicated no significant differences between the two groups. With both the quadratic and linear terms for PCA in the model, the chi-square for the difference between threshold and non-threshold patients was 5.634 (1 degree of freedom, p<0.02). The average difference in VEGF between the two groups was 86.8. Sepsis, IVH, BPD, and NEC were all tested in the model and found to be non-significant.

Conclusions These results demonstrate that serum VEGF is an important tool in identifying those patients at increased risk for developing neovascular ROP. Patients with stage 3 and threshold disease have a higher mean VEGF level, with a peak difference at about 36 weeks. These results also indicate serum VEGF levels that begin to decrease from 32 weeks PCA through term indicate a lower risk for developing ROP.

The complete disclosure of all patents, patent applications, and publications, and electronically available material (e.g., GenBank amino acid and nucleotide sequence submissions, and computer programs) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method for determining if a premature infant is at-risk of developing severe retinopathy of prematurity (ROP), the method comprising:
detecting the level of vascular endothelial growth factor (VEGF) in a first biological sample obtained from a premature infant;
detecting the level of VEGF in a second biological sample obtained from the premature infant, wherein the second biological sample is obtained after the first biological sample;
comparing the levels of VEGF in the first biological sample and the second biological sample, wherein a premature infant who is not at-risk of developing severe ROP has a lower level of VEGF in the second biological sample.

2. The method of claim 1 wherein the first and second biological sample is blood.

3. The method of claim 1 wherein the first biological sample is obtained when the premature infants' post-conceptual age is at about 32 weeks.

4. The method of claim 1 wherein the second biological sample is obtained about a week after the first biological sample is obtained.

5. A method for determining if a premature infant is at-risk of developing severe ROP, the method comprising:
detecting the level of VEGF in a first biological sample obtained from a premature infant;
detecting the level of VEGF in a second biological sample obtained from the premature infant;
comparing the levels of VEGF in the first biological sample and the second biological sample, wherein a premature infant who is at-risk of developing severe ROP has a higher level of VEGF in the second biological sample.

6. The method of claim 5 wherein the first and second biological sample is blood.

7. The method of claim 5 wherein the first biological sample is obtained when the premature infants' post-conceptual age is at about 32 weeks.

8. The method of claim 5 wherein the second biological sample is obtained about a week after the first biological sample is obtained.

9. A method for determining if a premature infant is at-risk of developing severe ROP, the method comprising:
detecting the level of VEGF in a first biological sample obtained from a premature infant, wherein the first biological sample is obtained from the premature infant by a post-conceptual age of about 35 weeks;
obtaining from the premature infant additional biological samples and detecting the level of VEGF in each of the additional biological samples, wherein each additional biological sample is obtained at weekly intervals following the first biological sample; and
comparing the levels of VEGF in each of the biological samples, wherein a premature infant who has a level of VEGF that is highest at about 36 weeks to about 37 weeks post-conception age is at-risk of developing severe ROP.

10. The method of claim 9 wherein the first and second biological sample is blood.

11. A method for determining if a premature infant is at-risk of developing severe ROP, the method comprising:
detecting the level of VEGF in a first biological sample obtained from a premature infant, wherein the first biological sample is obtained from the premature infant by a post-conceptual age of about 35 weeks;
obtaining from the premature infant additional biological samples and detecting the level of VEGF in each of the additional biological samples, wherein each additional biological sample is obtained at weekly intervals following the first biological sample; and
comparing the levels of VEGF in each of the biological samples, wherein a premature infant who has a level of VEGF that is not highest at about 36 weeks to about 37 weeks post-conception age is at-risk of developing severe ROP.

12. The method of claim 11 wherein the first and second biological sample is blood.

13. A kit for determining whether a premature infant is at-risk of developing severe ROP, the kit comprising reagents for measuring the level of VEGF in a biological sample and packaging material comprising instructions indicating how to determine whether a premature infant is at-risk for developing severe ROP.
